(19) United States
(12) Patent Application Publication
(10) Pub. No.: US 2026/0069232 A1
(43) Pub. Date: Mar. 12, 2026

(54) X-RAY IMAGING APPARATUS

(71) Applicant: SHIMADZU CORPORATION, Kyoto-shi (JP)

(72) Inventor: Masahiro TANAKA, Kyoto-shi (JP)

(21) Appl. No.: 19/307,278

(22) Filed: Aug. 22, 2025

(30) Foreign Application Priority Data
Sep. 12, 2024 (JP) 2024-158396

(51) Int. Cl. A61B 6/00; A61B 6/46; G06F 3/01; G06T 7/70

(52) U.S. Cl. CPC A61B 6/54; A61B 6/464; A61B 6/467; G06F 3/013; G06T 7/70; G06T 2207/30196

(57) ABSTRACT

An X-ray imaging apparatus 100 comprises an X-ray imaging unit 10 provided in an X-ray imaging room 92, a first console 20 for the X-ray imaging unit 10 provided in the X-ray imaging room 92, a second console 30 for the X-ray imaging unit 10 provided outside the X-ray imaging room 92, an analysis unit 40 that performs an analysis process on each of first detection information acquired by a first operator detection unit that detects a first operator 90a and second detection information acquired by a second operator detection unit that detects a second operator 90b to acquire first posture information 73 of the first operator 90a and second posture information 76 of the second operator 90b, and a control unit 50 that, based on the first posture information 73 and the second posture information 76, performs control to switch an operating authority 75 for enabling operation of the X-ray imaging unit 10 between the first console 20 and the second console 30.

### Technical Field

The present invention relates to an X-ray imaging apparatus, and more particularly to an X-ray imaging apparatus including a plurality of operation units for operating an X-ray imaging unit.

### Background Art

Conventionally, an X-ray imaging apparatus including a plurality of operation units for operating an X-ray imaging unit is known (see, for example, Patent Literature 1).

Patent Literature 1 above describes an X-ray imaging apparatus including an X-ray fluoroscopy table, an imaging unit, an operation panel, and an external operation unit. The X-ray fluoroscopy table includes a top plate on which a subject is placed. The imaging unit includes an X-ray source and an X-ray detector. The operation panel includes a plurality of operation input units for operating the top plate. The plurality of operation input units include a position adjustment button for the X-ray fluoroscopy table and an operating authority button. The external operation unit is provided at a location remote from the X-ray imaging apparatus and is configured to be capable of operating the X-ray imaging apparatus. In the configuration disclosed in Patent Literature 1, the X-ray imaging apparatus operates in response to an operation input from either the operation panel or the external operation unit that has the operating authority. The switching of the operating authority between the operation panel and the external operation unit is performed by operating the operating authority button.

### [Prior Art Documents]

### [Patent Literature]

[Patent Literature 1] Japanese Unexamined Patent Application Publication No. 2020-171484

### [Summary of Invention]

### [Problem to be Solved by the Invention]

However, in the case of switching the operating authority (operation right) between the operation panel (first console) and the external operation unit (second console) by a button operation, as in the X-ray imaging apparatus described in Patent Literature 1, the operator's operation for switching the operating authority becomes cumbersome. Therefore, there is a demand to suppress the cumbersomeness of the operator's operation when activating the operating authority.

This invention has been made to solve the above-described problems, and one object of the present invention is to provide an X-ray imaging apparatus capable of suppressing the cumbersomeness of an operator's operation when activating the operating authority.

### [Means for Solving the Problem]

An X-ray imaging apparatus according to one aspect of the present invention comprises an X-ray imaging unit provided in an X-ray imaging room and having an X-ray irradiation unit that irradiates X-rays, an X-ray detection unit that detects the X-rays irradiated from the X-ray irradiation unit, and a top plate; a first console provided in the X-ray imaging room and including a first operation unit for the X-ray imaging unit; a second console provided outside the X-ray imaging room and including a second operation unit for the X-ray imaging unit; a first operator detection unit that detects a first operator corresponding to the first console; a second operator detection unit that detects a second operator corresponding to the second console; an analysis unit that performs an analysis process on each of first detection information acquired by the first operator detection unit and second detection information acquired by the second operator detection unit to acquire first posture information of the first operator and second posture information of the second operator; and a control unit that, based on the first posture information and the second posture information, performs control to switch an operating authority for enabling operation of the X-ray imaging unit between the first console and the second console. The term "console" as used herein refers to a console-type input/output device having a plurality of operation units for operating the X-ray imaging unit.

### [Effects of the Invention]

Since the operating authority is switched between the first console and the second console based on the first posture information of the first operator and the second posture information of the second operator, the operation of the first console or the second console can be enabled without the first operator and the second operator performing an operation to switch the operating authority. As a result, it is possible to suppress the cumbersomeness of the operator's operation when activating the operating authority. Furthermore, since the operating authority is switched between the first console and the second console based on the first posture information of the first operator and the second posture information of the second operator, it is possible to suppress unnecessary switching of the operating authority when an operator crosses in front of a console.

### [Brief Description of Drawings]

FIG. 1 is a block diagram showing the overall configuration of an X-ray imaging apparatus according to an embodiment.

FIG. 2 is a schematic diagram showing the overall configuration of the X-ray imaging apparatus according to the embodiment.

FIG. 3 is a schematic diagram showing an example of a console according to the embodiment.

FIG. 4 is a schematic diagram for explaining a configuration in which a control unit and an analysis unit switch the operating authority to a first console according to the embodiment.

FIG. 5 is a schematic diagram for explaining a configuration in which the control unit and the analysis unit switch the operating authority to a second console according to the embodiment.

FIG. 6 is a schematic diagram for explaining a configuration in which the control unit and the analysis unit switch the operating authority when both operators are positioned in front of their respective consoles according to the embodiment.

FIG. 7 is a schematic diagram showing an example of a first password screen displayed on a first display unit by the X-ray imaging apparatus according to the embodiment.

FIG. 8 is a schematic diagram showing an example of a second password screen displayed on a second display unit by the X-ray imaging apparatus according to the embodiment.

FIG. 9 is a flowchart for explaining a process of switching the operating authority by the control unit and the analysis unit according to the embodiment.

FIG. 10 is a flowchart for explaining a process of switching the operating authority by the control unit when an operating authority button is operated, according to the embodiment.

FIG. 11 is a flowchart for explaining a process of displaying a password screen by the control unit and the analysis unit according to the embodiment.

### [Description of Embodiments]

Hereinafter, an embodiment embodying the present invention will be described based on the drawings.

### [Configuration of X-ray Imaging Apparatus]

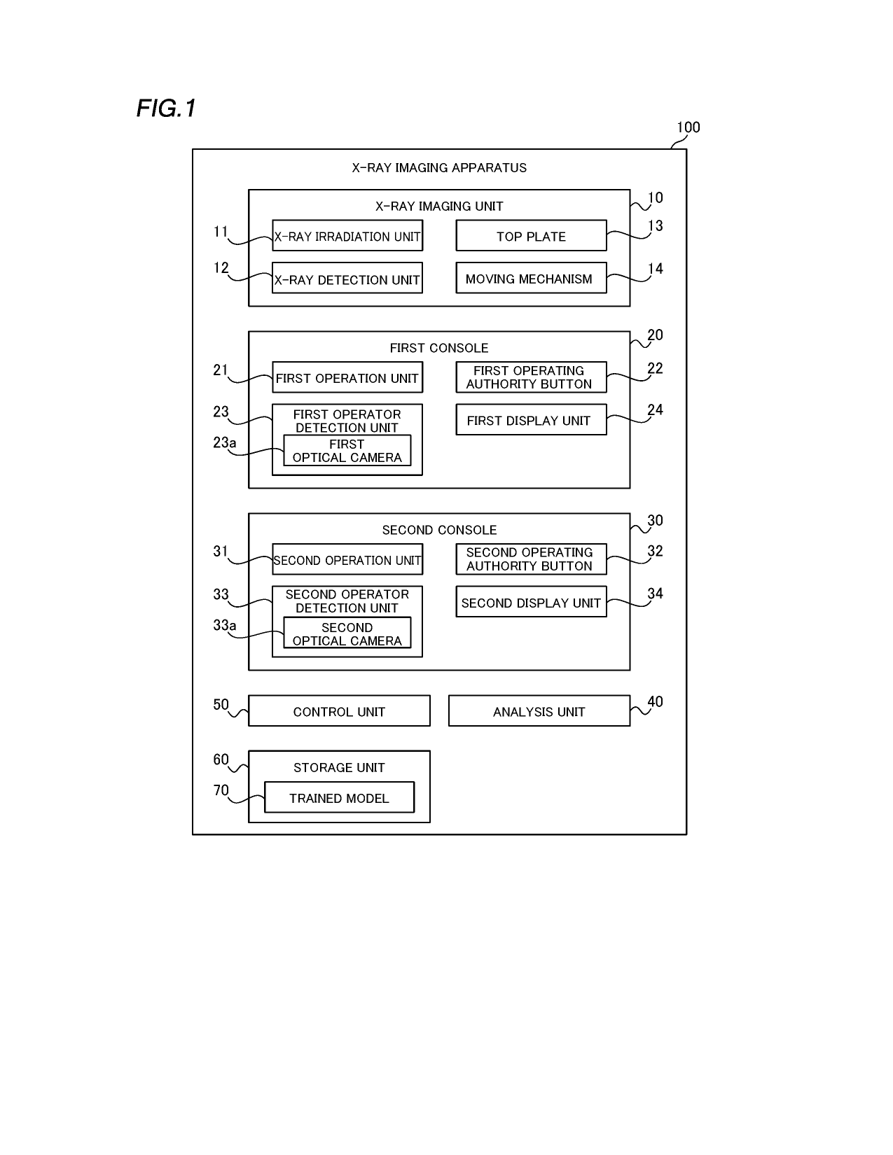

With reference to FIG. 1 to FIG. 11, the configuration of an X-ray imaging apparatus 100 according to an embodiment of the present invention will be described.

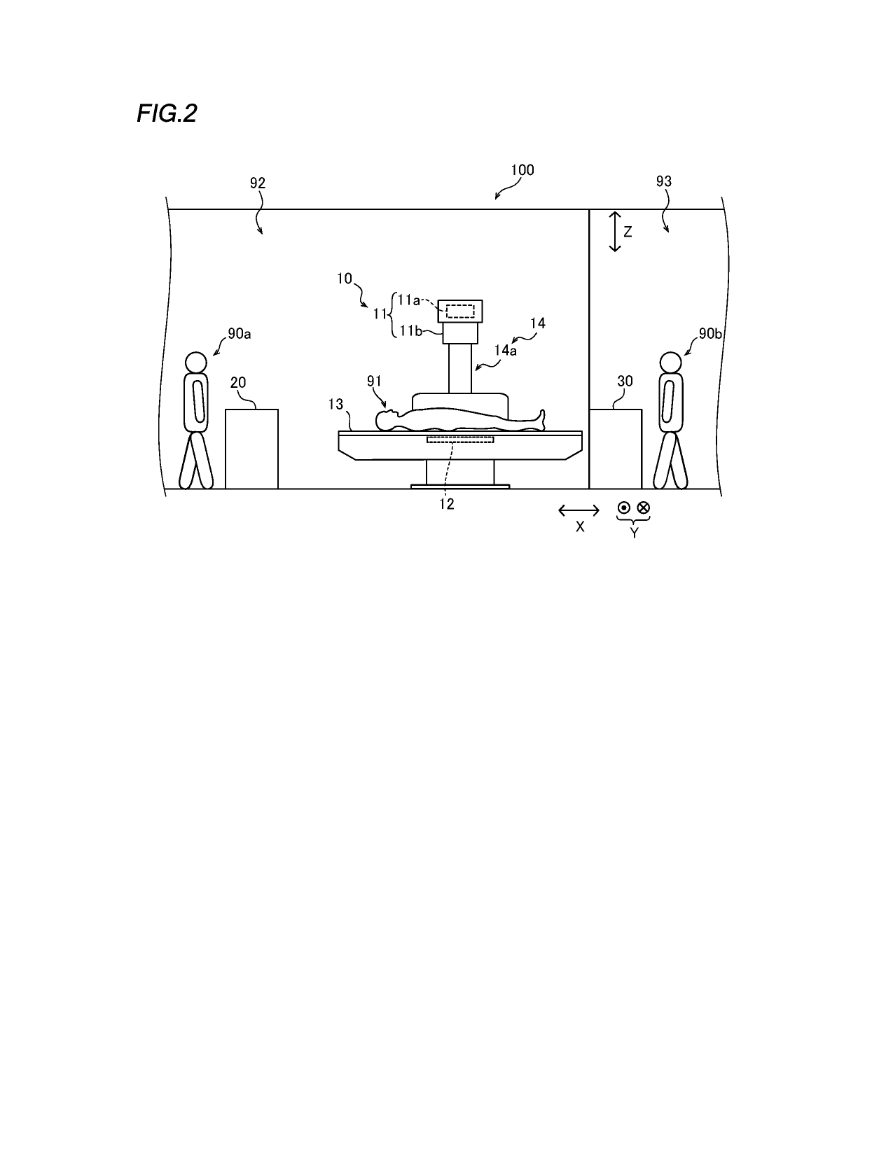

As shown in FIG. 1 and FIG. 2, the X-ray imaging apparatus 100 is a medical X-ray imaging apparatus (a so-called X-ray television system) that captures X-ray images (not shown) as moving images by continuously irradiating X-rays. The X-ray imaging apparatus 100 is configured to perform X-ray imaging of a subject 91, which is an imaging target.

The X-ray imaging apparatus 100 includes an X-ray imaging unit 10, a first console 20, a second console 30, an analysis unit 40, a control unit 50, and a storage unit 60.

The X-ray imaging unit 10 has an X-ray irradiation unit 11, an X-ray detection unit 12, a top plate 13, and a moving mechanism 14.

In the X-ray imaging apparatus 100, X-rays irradiated from the X-ray irradiation unit 11 are detected by the X-ray detection unit 12, whereby X-ray imaging of the subject 91 placed on the top plate 13 is performed.

The X-ray irradiation unit 11 irradiates X-rays. The X-ray irradiation unit 11 includes an X-ray tube 11a and a collimator unit 11b. The X-ray tube 11a is configured to irradiate X-rays when a predetermined voltage is applied. The collimator unit 11b has a plurality of position-adjustable shielding plates (collimator leaves). The collimator unit 11b is configured to define (adjust) the irradiation field of the X-rays irradiated from the X-ray tube 11a by shielding a part of the X-rays from the X-ray tube 11a. The collimator unit 11b is provided in the vicinity of the X-ray tube 11a in the X-ray irradiation direction of the X-ray tube 11a.

The X-ray detection unit 12 is configured to detect the X-rays irradiated from the X-ray irradiation unit 11. For example, the X-ray detection unit 12 is movably arranged below the top plate 13. The X-ray detection unit 12 includes a flat panel detector (FPD). The X-ray detection unit 12 outputs a signal based on the detected X-rays to the control unit 50.

The X-ray irradiation unit 11 is movably held within an X-ray imaging room 92 by the moving mechanism 14. Note that the vertical (perpendicular) direction is referred to as the Z direction, and two mutually orthogonal horizontal directions are referred to as the X direction and the Y direction. The moving mechanism 14 is configured to movably hold the X-ray irradiation unit 11. The moving mechanism 14 includes a support unit 14a that supports the X-ray irradiation unit 11. The moving mechanism 14 moves the X-ray irradiation unit 11 while the X-ray irradiation unit 11 and the top plate 13, on which the X-ray detection unit 12 is arranged, are facing each other. The moving mechanism 14, for example, integrally rotates the top plate 13 and the support unit 14a into a tilted state. The support unit 14a also changes the distance between the X-ray irradiation unit 11 and the top plate 13 on which the X-ray detection unit 12 is arranged. The moving mechanism 14 has, for example, a motor as a drive source.

The first console 20 includes a first operation unit 21, a first operating authority button 22, a first operator detection unit 23, and a first display unit 24.

The first operation unit 21 is an operation unit (console) for the X-ray imaging unit 10. Details of the first operation unit 21 will be described later.

The first operating authority button 22 is a button for switching an operating authority 75 between an active state and an inactive state, regardless of whether the operating authority 75 for operating the X-ray imaging unit 10 has been granted. In other words, the first operating authority button 22 is a button that can always be operated, regardless of the state of the operating authority 75. Each time the first operating authority button 22 is operated, the operating authority 75 of the first console 20 is switched between the active state and the inactive state. The first operating authority button 22 is also configured to be able to identify the state of the operating authority 75 of the first console 20. For example, the first operating authority button 22 is configured to be able to identify the state of the operating authority 75 by emitting light of a color corresponding to the state of the operating authority 75. Details of the configuration for switching the operating authority 75 by the first operating authority button 22 will be described later.

The first operator detection unit 23 is configured to detect a first operator 90a corresponding to the first console 20. In this embodiment, the first operator detection unit 23 is a first optical camera 23a that captures a first optical image 71. The first optical camera 23a is configured to be able to capture still images and moving images.

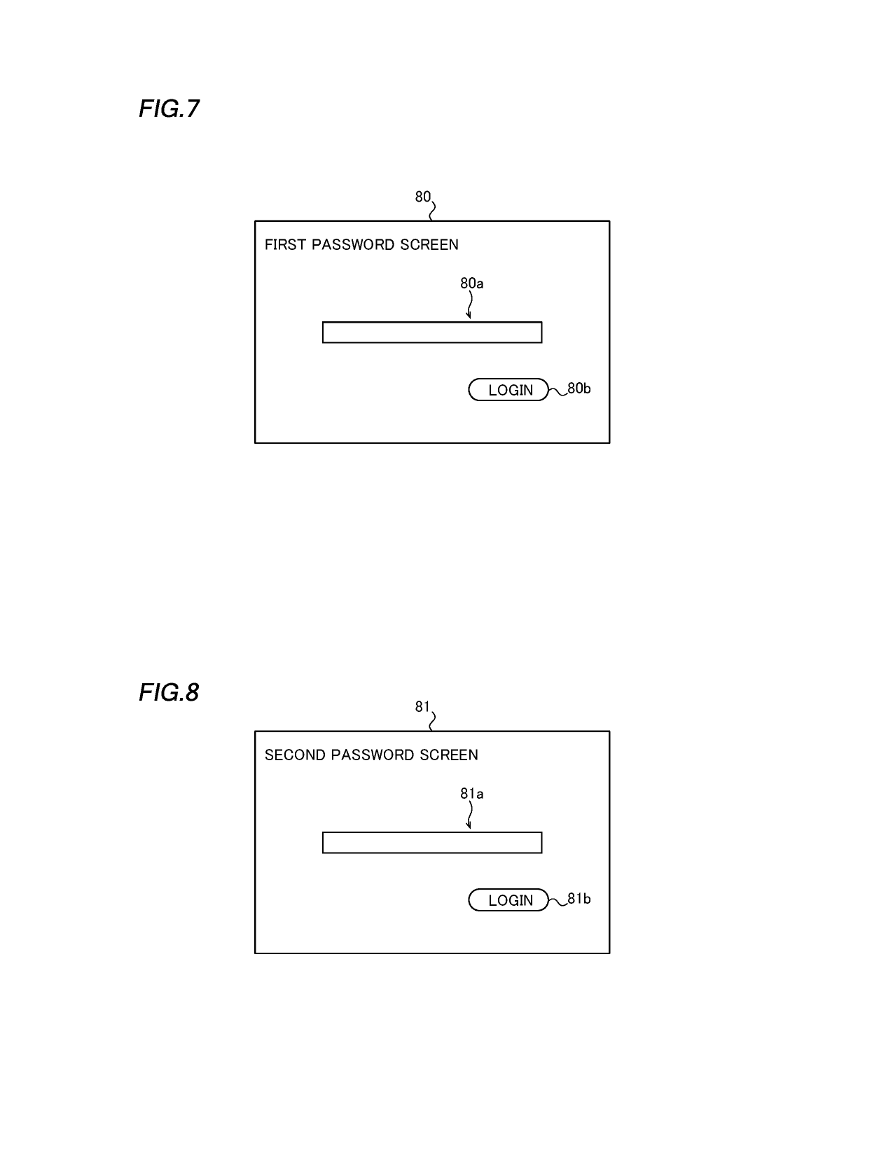

The first display unit 24 displays an operation screen (not shown) for when the first operator 90a operates the X-ray imaging unit 10. The first display unit 24 also displays a first password screen 80 (see FIG. 7) for the first operator 90a to enter a password to operate the X-ray imaging unit 10. The first display unit 24 is, for example, a display device such as a liquid crystal monitor.

The second console 30 includes a second operation unit 31, a second operating authority button 32, a second operator detection unit 33, and a second display unit 34.

The second operation unit 31 is an operation unit (console) for the X-ray imaging unit 10. Details of the second operation unit 31 will be described later.

The second operating authority button 32 is a button for switching the operating authority 75 between an active state and an inactive state, regardless of whether the operating authority 75 has been granted. In other words, the second operating authority button 32 is a button that can always be operated, regardless of the state of the operating authority 75. Each time the second operating authority button 32 is operated, the operating authority 75 of the second console 30 is switched between the active state and the inactive state. The second operating authority button 32 is also configured to be able to identify the state of the operating authority 75 of the second console 30. For example, the second operating authority button 32 is configured to be able to identify the state of the operating authority 75 by emitting light of a color corresponding to the state of the operating authority 75. Details of the configuration for switching the operating authority 75 by the second operating authority button 32 will be described later.

The second operator detection unit 33 is configured to detect a second operator 90b corresponding to the second console 30. In this embodiment, the second operator detection unit 33 is a second optical camera 33a that captures a second optical image 72. The second optical camera 33a is configured to be able to capture still images and moving images.

The second display unit 34 displays an operation screen (not shown) for when the second operator 90b operates the X-ray imaging unit 10. The second display unit 34 also displays a second password screen 81 (see FIG. 8) for the second operator 90b to enter a password to operate the X-ray imaging unit 10. The second display unit 34 is, for example, a display device such as a liquid crystal monitor.

The analysis unit 40 is configured to perform an analysis process on each of the first detection information and the second detection information to acquire first posture information 73 of the first operator 90a and second posture information 76 of the second operator 90b. The analysis unit 40 includes, for example, a GPU (Graphics Processing Unit) or an FPGA (Field-Programmable Gate Array) configured for image processing.

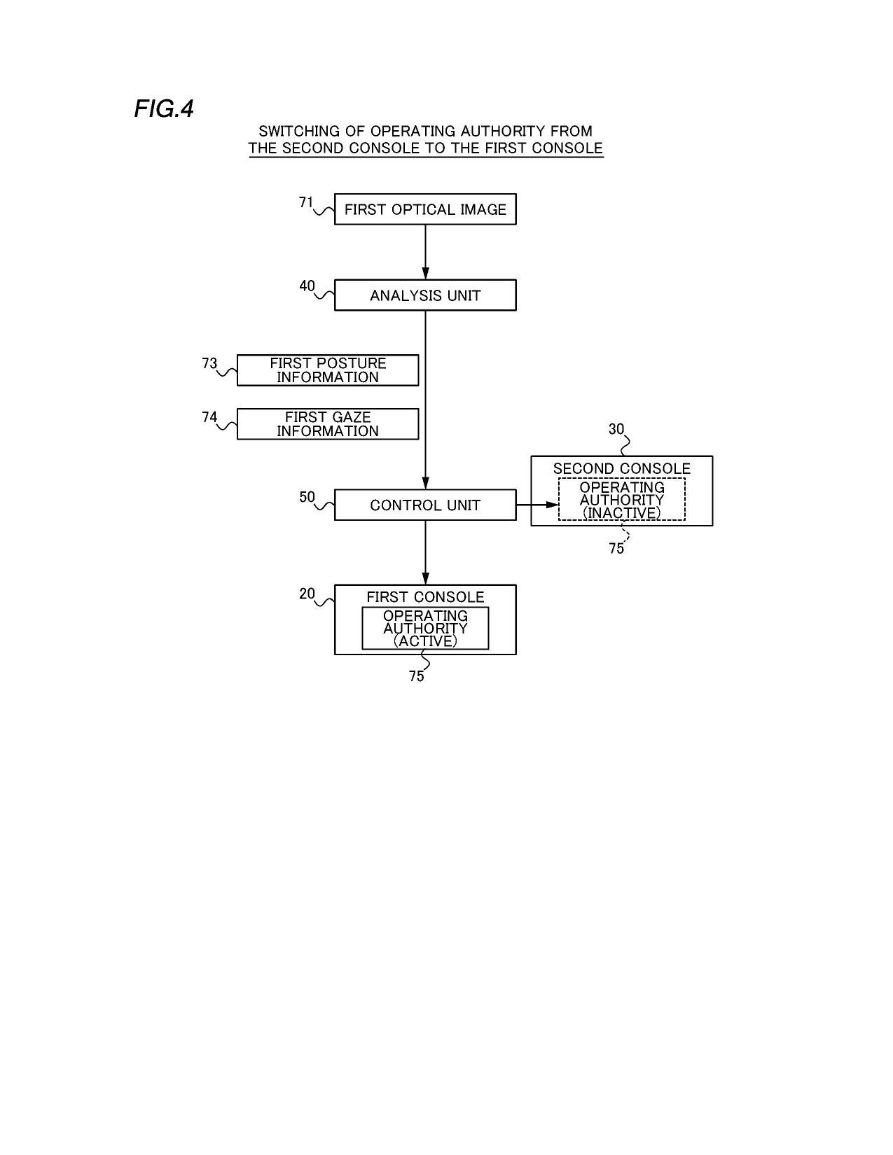

The first detection information is acquired by the first operator detection unit 23. Specifically, the first detection information is a first optical image 71 (see FIG. 4) acquired by the first optical camera 23a. In this embodiment, the first optical image 71 is a moving image.

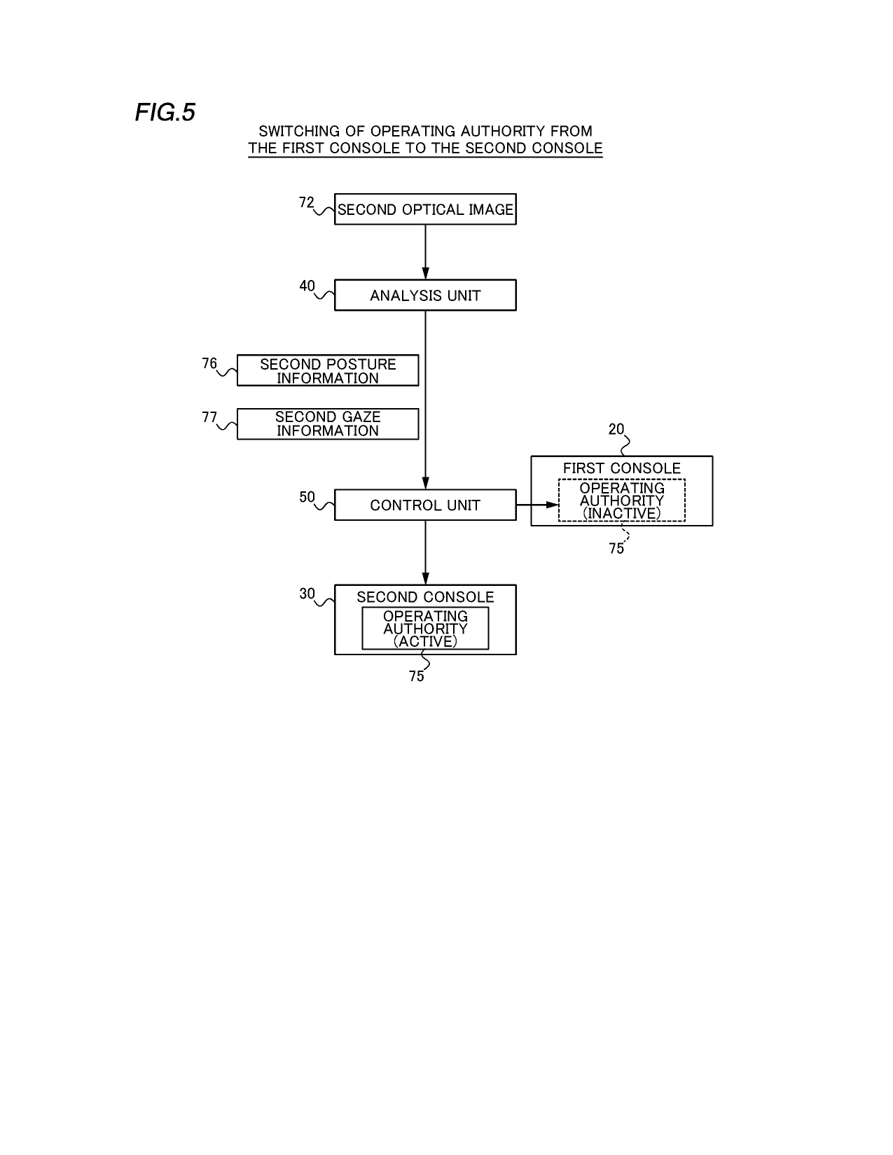

The second detection information is acquired by the second operator detection unit 33. Specifically, the second detection information is a second optical image 72 (see FIG. 5) acquired by the second optical camera 33a. In this embodiment, the second optical image 72 is a moving image.

The control unit 50 performs control of X-ray imaging by the X-ray irradiation unit 11 and the X-ray detection unit 12. The control unit 50 also performs control to switch the operating authority 75 between the first console 20 and the second console 30 based on the first posture information 73 (see FIG. 4) and the second posture information 76 (see FIG. 5). The control unit 50 includes a processor such as a CPU (Central Processing Unit) or circuitry, and memory such as ROM (Read Only Memory) and RAM (Random Access Memory). The control unit 50 performs control of X-ray imaging and control to switch the operating authority 75 based on parameters and various programs that are preset and stored in the storage unit 60. The control unit 50 also performs control of the display of the first password screen 80 and the second password screen 81. Details of the control to switch the operating authority 75 and the control of the display of the password screen performed by the control unit 50 will be described later.

The storage unit 60 stores a trained model 70 that the analysis unit 40 uses for analysis. The storage unit 60 also stores various programs and parameters executed by the control unit 50. The storage unit 60 is, for example, a non-volatile storage device such as an HDD (Hard Disk Drive) or an SSD (Solid State Drive).

The trained model 70 is created by training it to output posture information and gaze information of a person appearing in an optical image when the optical image is input. Specifically, the trained model 70 is created by training it to output posture information on whether the person in the optical image is facing forward, and gaze information on whether the gaze of the person in the optical image is directed toward the display unit. The trained model 70 is created in advance and stored in the storage unit 60.

As shown in FIG. 2, the X-ray imaging unit 10 is provided in the X-ray imaging room 92. The first console 20 is also provided in the X-ray imaging room 92. The second console 30 is provided outside the X-ray imaging room 92. The second console 30 is provided, for example, in an operation room 93 outside the X-ray imaging room 92.

The first console 20 is used by the first operator 90a to operate the X-ray imaging unit 10 within the X-ray imaging room 92.

The second console 30 is used by the second operator 90b to operate the X-ray imaging unit 10 within the operation room 93.

### (Console)

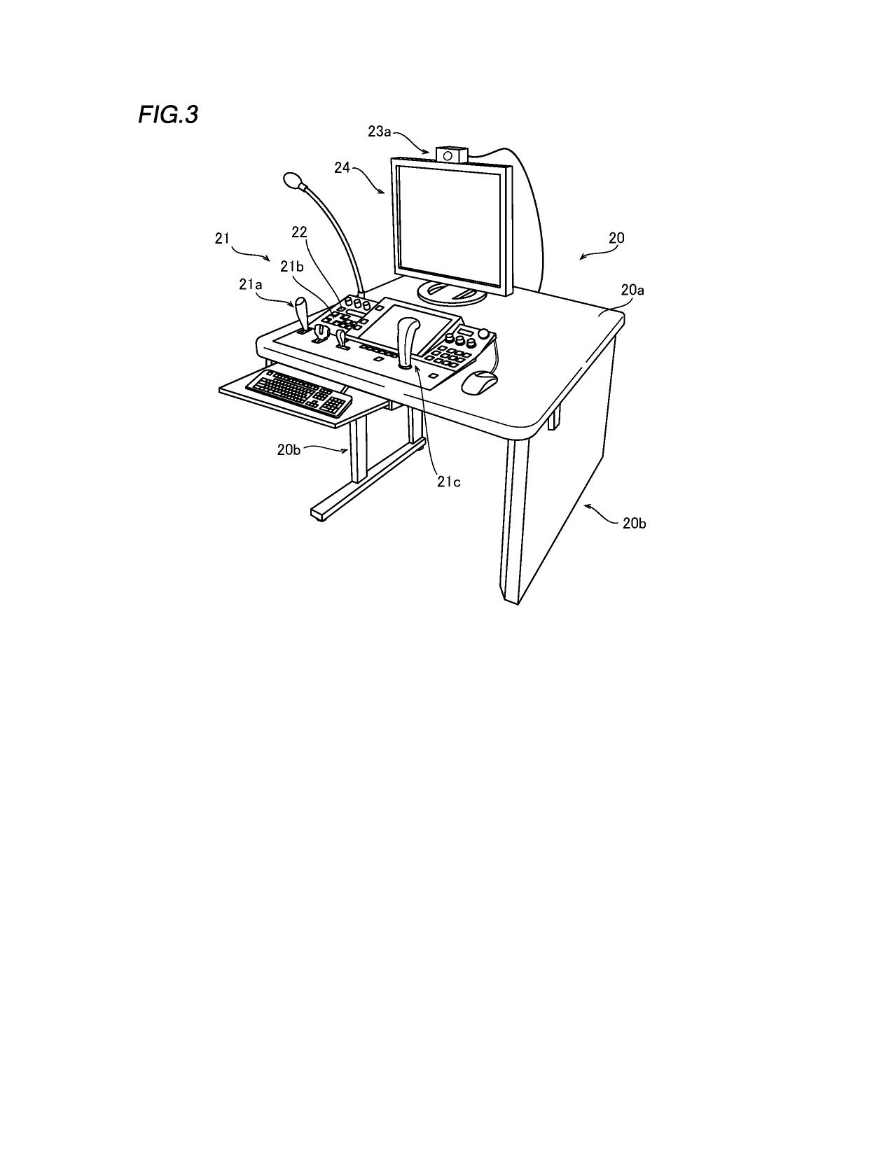

Next, the configuration of the console will be described with reference to FIG. 3. The first console 20 (see FIG. 2) and the second console 30 (see FIG. 2) differ only in whether they are provided inside or outside the X-ray imaging room 92, and their substantial configurations are the same. Therefore, in FIG. 3, the first console 20 is described as a representative of the two consoles, and a detailed description of the second console 30 is omitted.

As shown in FIG. 3, the first console 20 has a top plate 20a and a support portion 20b.

The top plate 20a is provided with the first operation unit 21 and the first operating authority button 22. The first display unit 24 is placed on the top plate 20a. The support portion 20b is a member that supports the top plate 20a.

The first operation unit 21 includes top plate operation units 21a and 21b for operating the top plate 13 (see FIG. 2), and an imaging unit operation unit 21c for operating the X-ray imaging unit 10 (see FIG. 2).

The top plate operation unit 21a is an operation unit for rotating the top plate 13. The top plate operation unit 21a is, for example, a joystick.

The top plate operation unit 21b is an operation unit for raising and lowering the top plate 13. The top plate operation unit 21b is, for example, a push button.

The imaging unit operation unit 21c is an operation unit for moving the X-ray imaging unit 10. The imaging unit operation unit 21c is, for example, a joystick.

The first optical camera 23a is provided on the upper part of the first display unit 24. The first optical camera 23a is configured to be detachable.

In the second console 30 as well, a second display unit 34 is placed on a top plate, and a second optical camera 33a is provided on the upper part of the second display unit 34. The second optical camera 33a is also configured to be detachable, similar to the first optical camera 23a. This allows the present invention to be easily applied to X-ray imaging apparatuses already installed in facilities such as hospitals.

### (Switching of Operating Authority to First Console)

Next, with reference to FIG. 4, a configuration in which the analysis unit 40 and the control unit 50 switch the operating authority 75 to the first console 20 will be described.

In the configuration for switching the operating authority 75 to the first console 20, the analysis unit 40 acquires the first optical image 71 from the first optical camera 24a. Then, the analysis unit 40 is configured to acquire the first posture information 73 of the first operator 90a appearing in the first optical image 71 by analyzing the first optical image 71 using the trained model 70 or the like stored in the storage unit 60. In this embodiment, the analysis unit 40, by analyzing the first optical image 71, acquires first gaze information 74 of the first operator 90a along with the first posture information 73.

The first posture information 73 is information on whether the first operator 90a is facing the front of the first console 20. Specifically, the first posture information 73 is numerical information representing the degree of certainty that the first operator 90a is facing the front of the first console 20. A larger numerical value of the first posture information 73 means that it is more likely that the first operator 90a is facing the front of the first console 20.

The first gaze information 74 is information on whether the first operator 90a is looking at the first display unit 24. Specifically, the first gaze information 74 is numerical information representing the degree of certainty that the first operator 90a is looking at the first display unit 24. A larger numerical value of the first gaze information 74 means that it is more likely that the first operator 90a is looking at the first display unit 24.

The analysis unit 40 outputs the acquired first posture information 73 and first gaze information 74 to the control unit 50.

When the first posture information 73 is information indicating that the first operator 90a is facing the front of the first console 20, the control unit 50 performs control to switch the operating authority 75 to the first console 20. In this embodiment, the control unit 50 performs control to switch the operating authority 75 to the first console 20 when the gaze of the first operator 90a is directed toward the first display unit 24. That is, the control unit 50 switches the operating authority 75 to the first console 20 when the first operator 90a is facing the front of the first console 20 and the gaze of the first operator 90a is directed toward the first display unit 24.

Specifically, the control unit 50 switches the operating authority 75 to the first console 20 when a state in which the first posture information 73 is equal to or greater than a predetermined first threshold and the first gaze information 74 is equal to or greater than a predetermined second threshold is maintained for a predetermined time. The predetermined time is, for example, one second.

By switching the operating authority 75 to an active state, the first operation unit 21 becomes operable. When the operating authority 75 is in an inactive state, the first operation unit 21 is in an inoperable state.

When the operating authority 75 is switched from the second console 30 to the first console 20, the control unit 50 switches the operating authority 75 of the second console 30 to an inactive state. When the operating authority 75 of the first console 20 is switched to an active state from a state where the operating authorities 75 of both consoles are inactive, the control unit 50 does not change the operating authority 75 of the second console 30. In FIG. 4, the active state of the operating authority 75 is illustrated by a solid line, and the inactive state is illustrated by a broken line.

The control unit 50 also performs control to switch the operating authority 75 to the first console 20 when an operation to activate the operating authority 75 is performed by the first operating authority button 22 in a state where the operating authority 75 is not granted to the first console 20.

### (Switching of Operating Authority to Second Console)

Next, with reference to FIG. 5, a configuration in which the analysis unit 40 and the control unit 50 switch the operating authority 75 to the second console 30 will be described.

In the configuration for switching the operating authority 75 to the second console 30, the analysis unit 40 acquires the second optical image 72 from the second optical camera 34a. Then, the analysis unit 40 is configured to acquire the second posture information 76 of the second operator 90b appearing in the second optical image 72 by analyzing the second optical image 72 using the trained model 70 or the like stored in the storage unit 60. In this embodiment, the analysis unit 40, by analyzing the second optical image 72, acquires second gaze information 77 of the second operator 90b along with the second posture information 76.

The second posture information 76 is information on whether the second operator 90b is facing the front of the second console 30. Specifically, the second posture information 76 is numerical information representing the degree of certainty that the second operator 90b is facing the front of the second console 30. A larger numerical value of the second posture information 76 means that it is more likely that the second operator 90b is facing the front of the second console 30.

The second gaze information 77 is information on whether the second operator 90b is looking at the second display unit 34. Specifically, the second gaze information 77 is numerical information representing the degree of certainty that the second operator 90b is looking at the second display unit 34. A larger numerical value of the second gaze information 77 means that it is more likely that the second operator 90b is looking at the second display unit 34.

The analysis unit 40 outputs the acquired second posture information 76 and second gaze information 77 to the control unit 50.

When the second posture information 76 is information indicating that the second operator 90b is facing the front of the second console 30, the control unit 50 performs control to switch the operating authority 75 to the second console 30. In this embodiment, the control unit 50 performs control to switch the operating authority 75 to the second console 30 when the gaze of the second operator 90b is directed toward the second display unit 34. That is, the control unit 50 switches the operating authority 75 to the second console 30 when the second operator 90b is facing the front of the second console 30 and the gaze of the second operator 90b is directed toward the second display unit 34.

Specifically, the control unit 50 switches the operating authority 75 to the second console 30 when a state in which the second posture information 76 is equal to or greater than a predetermined first threshold and the second gaze information 77 is equal to or greater than a predetermined second threshold is maintained for a predetermined time.

When the operating authority 75 is switched from the first console 20 to the second console 30, the control unit 50 switches the operating authority 75 of the first console 20 to an inactive state. When the operating authority 75 of the second console 30 is switched to an active state from a state where the operating authorities 75 of both consoles are inactive, the control unit 50 does not change the operating authority 75 of the first console 20. In FIG. 5, the active state of the operating authority 75 is illustrated by a solid line, and the inactive state is illustrated by a broken line.

By switching the operating authority 75 to an active state, the second operation unit 31 becomes operable. When the operating authority 75 is in an inactive state, the second operation unit 31 is in an inoperable state.

The control unit 50 also performs control to switch the operating authority 75 to the second console 30 when an operation to activate the operating authority 75 is performed by the second operating authority button 32 in a state where the operating authority 75 is not granted to the second console 30.

### (Control When Both First and Second Operators are in Front of Consoles)

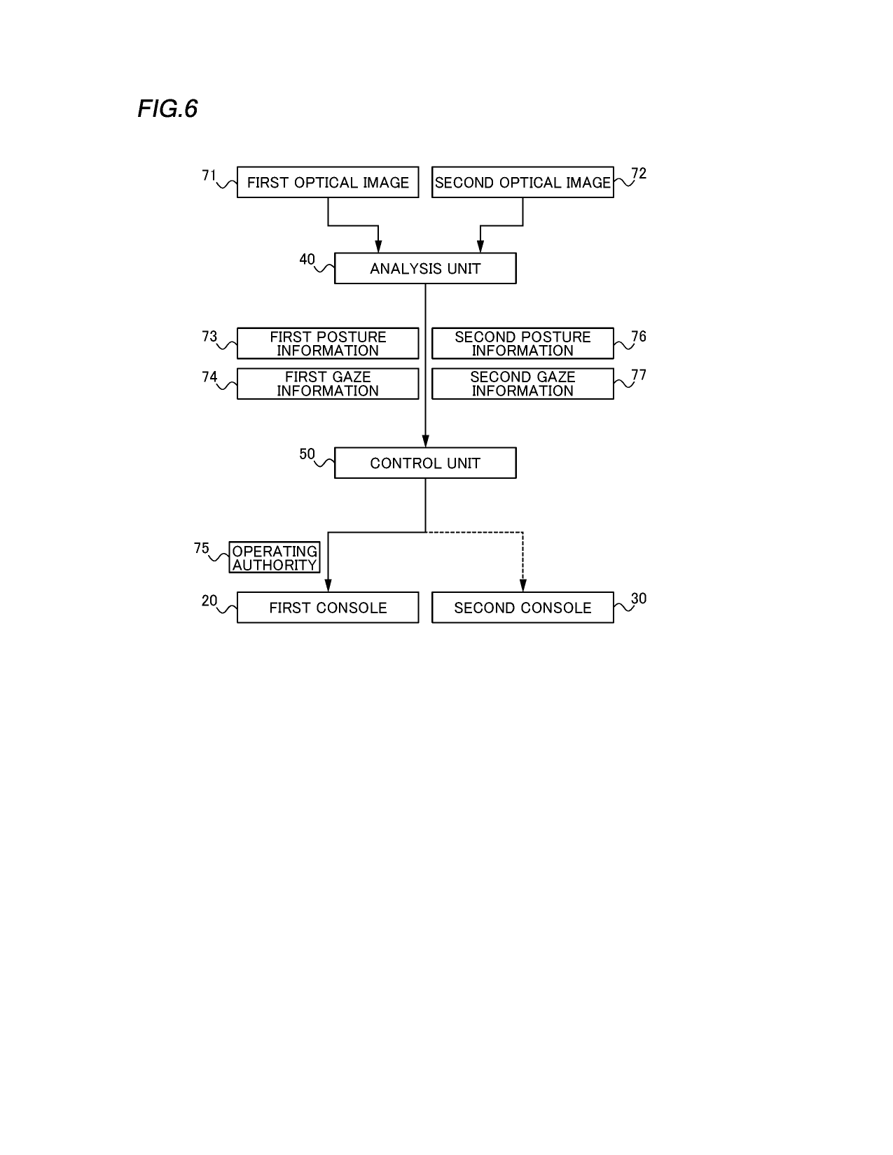

Next, with reference to FIG. 6, control performed by the analysis unit 40 and the control unit 50 to switch the operating authority 75 when both the first operator 90a and the second operator 90b are in front of their respective consoles will be described.

When both the first operator 90a and the second operator 90b are in front of their consoles, the first optical image 71 is input from the first optical camera 24a to the analysis unit 40. The second optical image 72 is also input from the second optical camera 34a to the analysis unit 40.

The analysis unit 40 analyzes each of the first optical image 71 and the second optical image 72 to acquire the first posture information 73, the first gaze information 74, the second posture information 76, and the second gaze information 77. The analysis unit 40 then outputs the acquired first posture information 73, first gaze information 74, second posture information 76, and second gaze information 77 to the control unit 50. The acquisition of the first posture information 73, the first gaze information 74, the second posture information 76, and the second gaze information 77 by the analysis unit 40 is performed sequentially in parallel at predetermined timings. Then, upon acquiring the first posture information 73, the first gaze information 74, the second posture information 76, and the second gaze information 77, the analysis unit 40 sequentially outputs them to the control unit 50.

When the first operator 90a is facing the front of the first console 20 and the second operator 90b is facing the front of the second console 30, the control unit 50 performs control to switch the operating authority 75 to the console for which the operator faced the front of the console at an earlier timing, among the first console 20 and the second console 30.

The control unit 50 determines which operator faced the front of their console at an earlier timing by determining which is earlier: the timing at which the state where the first posture information 73 and the first gaze information 74 are equal to or greater than a predetermined first threshold has been maintained for a predetermined time, or the timing at which the state where the second posture information 76 and the second gaze information 77 are equal to or greater than a predetermined first threshold has been maintained for a predetermined time. In other words, the control unit 50 switches the operating authority 75 to the console for which it determined to grant the operating authority 75 earlier, between the timing it determined to grant the operating authority 75 to the first console 20 and the timing it determined to grant the operating authority 75 to the second console 30.

The example shown in FIG. 6 illustrates a case where the timing at which it was determined to grant the operating authority 75 to the first console 20 is earlier than the timing at which it was determined to grant the operating authority 75 to the second console 30. Therefore, the control unit 50 switches the operating authority 75 of the first console 20 to an active state and switches the operating authority 75 of the second console 30 to an inactive state. In FIG. 6, the fact that the operating authority 75 of the second console 30 is not switched to the active state is represented by showing the arrow drawn from the control unit 50 to the second console 30 as a broken line.

After the first operator 90a is facing the front of the first console 20 and the second operator 90b is facing the front of the second console 30, if either the first operator 90a or the second operator 90b moves away from the front of the console and a predetermined time (e.g., 10 seconds) has elapsed, the control unit 50 performs control to switch the operating authority 75 to the console that an operator is facing, among the first console 20 and the second console 30. For example, after the first operator 90a is facing the front of the first console 20 and the second operator 90b is facing the front of the second console 30, if the first operator 90a moves away from the front of the first console 20 for a predetermined time, the control unit 50 switches the operating authority 75 to the second console 30.

### (Password Screen)

Next, with reference to FIG. 7 and FIG. 8, the first password screen 80 displayed on the first display unit 24 and the second password screen 81 displayed on the second display unit 34 will be described.

The first password screen 80 shown in FIG. 7 is a screen displayed on the first display unit 24 when operating the X-ray imaging unit 10 and the top plate 13 at the first console 20. The first password screen 80 displays a password input field 80a and a login button 80b.

The password input field 80a is a text box. The login button 80b is a GUI (Graphical User Interface) push button.

When the first operator 90a enters a password in the password input field 80a and the login button 80b is operated (pressed), the control unit 50 determines whether the entered password is correct. If the entered password is correct, the control unit 50 enables the operation of the first console 20.

The control unit 50 displays the first password screen 80 when the first console 20 is operated for the first time after the X-ray imaging apparatus 100 is started. The control unit 50 may also display the first password screen 80 if the first console 20 is not operated for a predetermined time, and enable the operation of the first console 20 upon password entry.

Here, if the first password screen 80 remains displayed on the first display unit 24, there is a possibility that the password could be entered by remote control from outside the X-ray imaging room 92, and the X-ray imaging unit 10 and the top plate 13 could be operated. Therefore, in this embodiment, the control unit 50 is configured to perform control not to display the first password screen 80 on the first display unit 24 in a state where there is no first operator 90a facing the front of the first console 20.

Specifically, the control unit 50 determines whether the first operator 90a is facing the front of the first console 20 based on the first posture information 73. Then, when the first posture information 73 is equal to or greater than a predetermined third threshold, the control unit 50 causes the first password screen 80 to be displayed. When the first posture information 73 is smaller than the predetermined third threshold, the control unit 50 performs control not to display the first password screen 80. Note that, when displaying the first password screen 80, it is not necessary for the first operator 90a to be strictly facing the front of the first console 20, as long as the first operator 90a is positioned in front of the first console 20. Therefore, the third threshold may be set to a value smaller than the first threshold. In the determination of whether to display the first password screen 80, it is not necessary for the control unit 50 to use the first gaze information 74.

The second password screen 81 shown in FIG. 8 is a screen displayed on the second display unit 34 when operating the X-ray imaging unit 10 and the top plate 13 at the second console 30. The second password screen 81 displays a password input field 81a and a login button 81b.

The password input field 81a is a text box. The login button 81b is a GUI push button.

When the second operator 90b enters a password in the password input field 81a and the login button 81b is operated (pressed), the control unit 50 determines whether the entered password is correct. If the entered password is correct, the control unit 50 enables the operation of the second console 30.

The control unit 50 displays the second password screen 81 when the second console 30 is operated for the first time after the X-ray imaging apparatus 100 is started. The control unit 50 may also display the second password screen 81 if the second console 30 is not operated for a predetermined time, and enable the operation of the second console 30 upon password entry.

The control unit 50 is also configured to perform control not to display the second password screen 81 on the second display unit 34 in a state where there is no second operator 90b facing the front of the second console 30.

Specifically, the control unit 50 determines whether the second operator 90b is facing the front of the second console 30 based on the second posture information 76. Then, when the second posture information 76 is equal to or greater than a predetermined third threshold, the control unit 50 causes the second password screen 81 to be displayed. When the second posture information 76 is smaller than the predetermined third threshold, the control unit 50 performs control not to display the second password screen 81. In the determination of whether to display the second password screen 81, it is not necessary for the control unit 50 to use the second gaze information 77.

### (Operating Authority Switching Process)

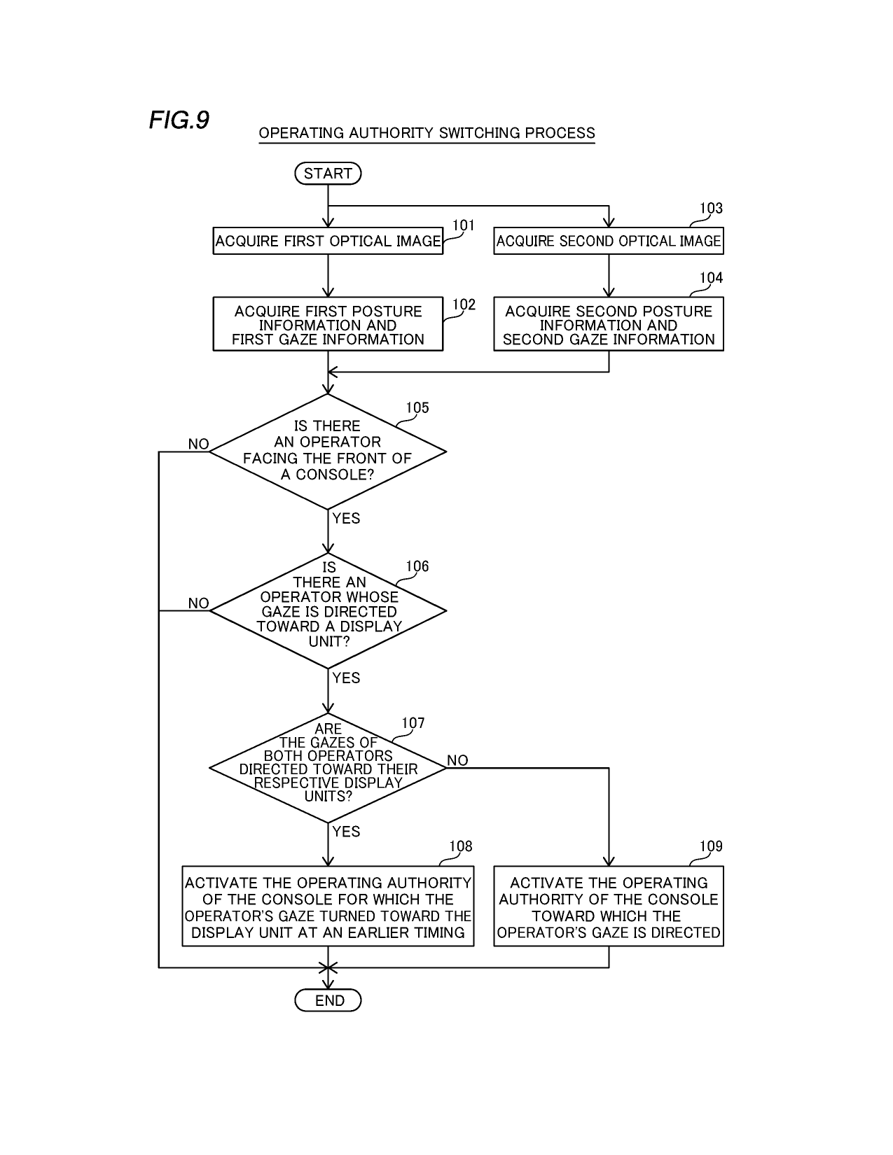

Next, with reference to FIG. 9, a process in which the analysis unit 40 and the control unit 50 switch the operating authority 75 between the first console 20 and the second console 30 will be described.

In step 101, the analysis unit 40 acquires the first optical image 71 from the first optical camera 24a.

Next, in step 102, the analysis unit 40 inputs the first optical image 71 into the trained model 70 and acquires the first posture information 73 and the first gaze information 74.

In step 103, the analysis unit 40 acquires the second optical image 72 and the second posture information 76 from the second optical camera 34a.

The processing of steps 101 and 102 and the processing of steps 103 and 104 are performed in parallel.

Next, in step 105, the control unit 50 determines whether there is an operator facing the front of a console. Specifically, the control unit 50 determines whether the first operator 90a is facing the front of the first console 20 based on the first posture information 73. The control unit 50 also determines whether the second operator 90b is facing the front of the second console 30 based on the second posture information 76. If at least one of the first operator 90a and the second operator 90b is facing the front of a console, the process proceeds to step 106. If neither the first operator 90a nor the second operator 90b is facing the front of a console, the process ends.

If the process proceeds from step 105 to step 106, then in step 106, the control unit 50 determines whether there is an operator whose gaze is directed toward a display unit. Specifically, the control unit 50 determines whether the gaze of the first operator 90a is directed toward the first display unit 24 based on the first gaze information 74. The control unit 50 also determines whether the gaze of the second operator 90b is directed toward the second display unit 34 based on the second gaze information 77. If the gaze of at least one of the first operator 90a and the second operator 90b is directed toward a display unit, the process proceeds to step 107. If the gazes of both the first operator 90a and the second operator 90b are not directed toward a display unit, the process ends.

If the process proceeds from step 106 to step 107, then in step 107, the control unit 50 determines whether the gazes of the first operator 90a and the second operator 90b are directed toward their respective display units. If the gazes of both the first operator 90a and the second operator 90b are directed toward their respective display units, the process proceeds to step 108. If the gaze of either one of the first operator 90a and the second operator 90b is not directed toward a display unit, the process proceeds to step 109.

If the process proceeds from step 107 to step 108, the control unit 50, as shown in FIG. 6, activates the operating authority 75 of the console for which the operator's gaze turned toward the display unit at an earlier timing. The control unit 50 also deactivates the operating authority 75 of the console for which the operator's gaze turned toward the display unit at a later timing. Thereafter, the process ends.

If the process proceeds from step 107 to step 109, the control unit 50, as shown in FIG. 4 or FIG. 5, activates the operating authority 75 of the console toward which the operator's gaze is directed. The control unit 50 also deactivates the operating authority 75 of the console toward which the operator's gaze is not directed. Thereafter, the process ends.

### (Operating Authority Switching Process When Operating Authority Button is Operated)

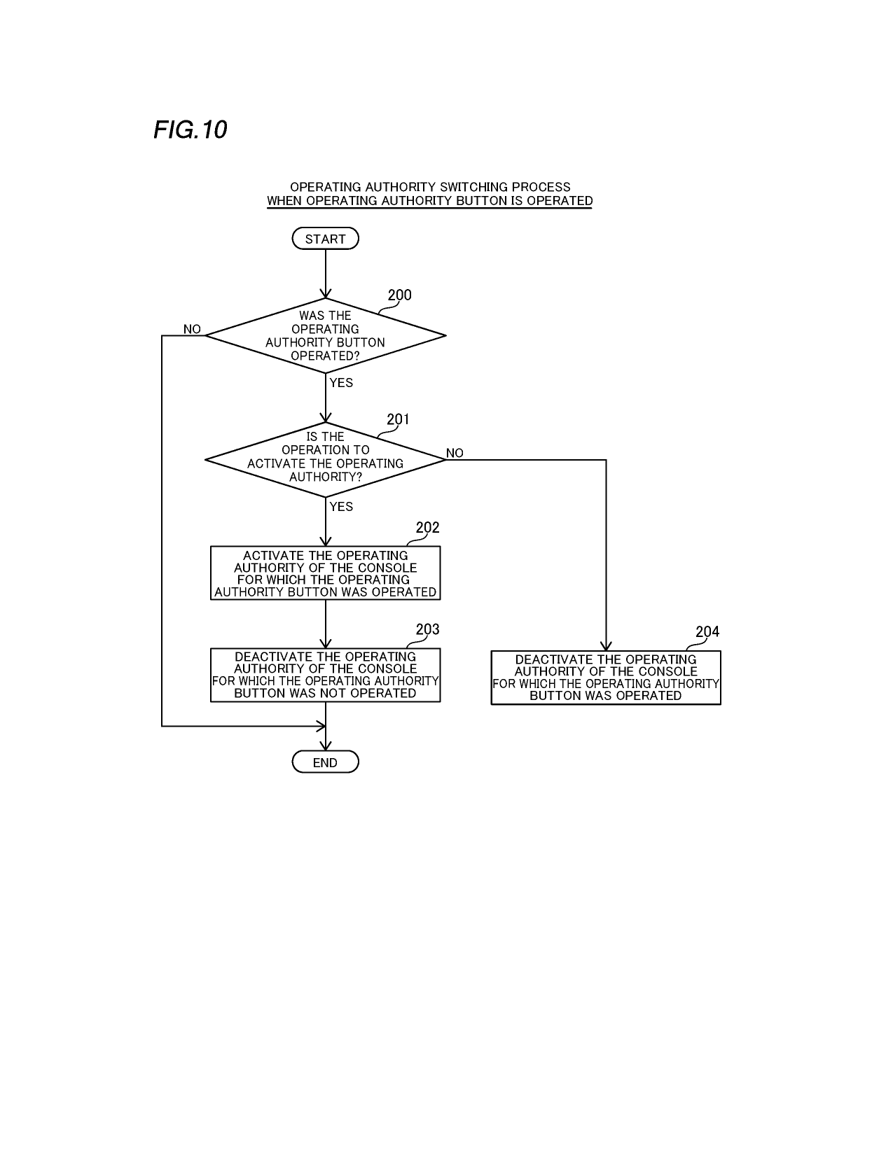

Next, with reference to FIG. 10, the process performed when the first operating authority button 22 or the second operating authority button 32 is operated will be described.

In step 200, the control unit 50 determines whether the first operating authority button 22 or the second operating authority button 32 has been operated. If the first operating authority button 22 or the second operating authority button 32 has been operated, the process proceeds to step 201. If neither the first operating authority button 22 nor the second operating authority button 32 has been operated, the process ends.

In step 201, the control unit 50 determines whether the operation is to activate the operating authority 75. If the operation is to activate the operating authority 75, the process proceeds to step 202. If the operation is not to activate the operating authority 75, the process proceeds to step 204.

If the process proceeds from step 201 to step 202, then in step 202, the control unit 50 activates the operating authority 75 of the console for which the operating authority button was operated. Specifically, if the first operating authority button 22 was operated, the control unit 50 activates the operating authority 75 of the first console 20. If the second operating authority button 32 was operated, the control unit 50 activates the operating authority 75 of the second console 30.

Next, in step 203, the control unit 50 deactivates the operating authority 75 of the console for which the operating authority button was not operated. Specifically, if the control unit 50 activated the operating authority 75 of the first console 20 in the processing of step 202, it deactivates the operating authority 75 of the second console 30. If the control unit 50 activated the operating authority 75 of the second console 30 in the processing of step 202, it deactivates the operating authority 75 of the first console 20. Thereafter, the process ends.

If the process proceeds from step 201 to step 204, then in step 204, the control unit 50 deactivates the operating authority 75 of the console for which the operating authority button was operated. Specifically, if an operation to deactivate the operating authority 75 was performed by the first operating authority button 22, the control unit 50 deactivates the operating authority 75 of the first console 20. If an operation to deactivate the operating authority 75 was performed by the second operating authority button 32, the control unit 50 deactivates the operating authority 75 of the second console 30. The control unit 50 does not perform an operation to activate the operating authority 75 of the console for which it did not deactivate the operating authority 75. Thereafter, the process ends.

### (Password Screen Display Process)

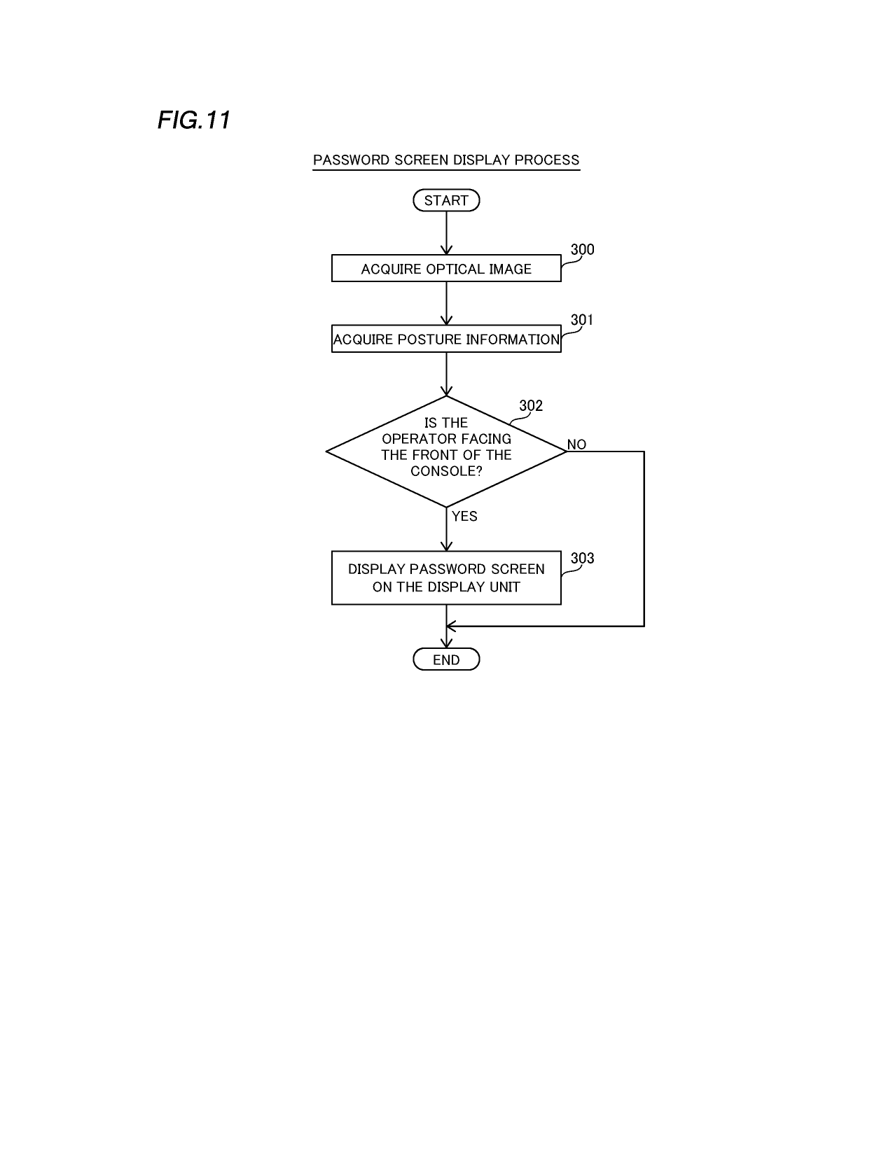

Next, with reference to FIG. 11, a process in which the analysis unit 40 and the control unit 50 display a password screen will be described.

In step 300, the analysis unit 40 acquires an optical image. Specifically, the analysis unit 40 acquires the first optical image 71 and the second optical image 72.

Next, in step 301, the analysis unit 40 acquires posture information. Specifically, the analysis unit 40 inputs the first optical image 71 into the trained model 70 and acquires the first posture information 73. The analysis unit 40 also inputs the second optical image 72 into the trained model 70 and acquires the second posture information 76.

Next, in step 302, the control unit 50 determines whether an operator is facing the front of a console. Specifically, the control unit 50 determines whether the first operator 90a is facing the front of the first console 20 and whether the second operator 90b is facing the front of the second console 30. If there is an operator facing the front of a console, the process proceeds to step 303. If there is no operator facing the front of a console, the process ends.

If the process proceeds from step 302 to step 303, then in step 303, the control unit 50 displays the password screen on the display unit. Specifically, if the first operator 90a is facing the front of the first console 20, the control unit 50 displays the first password screen 80 on the first display unit 24. If the second operator 90b is facing the front of the second console 30, the control unit 50 displays the second password screen 81 on the second display unit 34. Thereafter, the process ends.

### [Variations]

It should be understood that the embodiment disclosed this time is exemplary in all respects and not restrictive. The scope of the present invention is indicated by the claims rather than by the description of the embodiment above, and all modifications (variations) within the meaning and scope equivalent to the claims are intended to be included therein.

For example, the control unit may switch the operating authority between the first console and the second console without being based on posture information. For example, the control unit may switch the operating authority between the first console and the second console based only on gaze information.

Also, when both the first operator and the second operator face their respective consoles, the control unit does not have to switch the operating authority to the one who faced the console earlier. For example, a priority may be set for each console, and the operating authority may be switched to the console with the higher priority.

Also, after both the first operator and the second operator have faced their respective consoles, the operating authority does not have to be switched even if one of the operators moves away from the front of the console. In this case, the operating authority can be switched by the operating authority button.

Also, the first operator detection unit and the second operator detection unit do not have to be optical cameras as long as they can acquire the operator's posture information. For example, the first operator detection unit and the second operator detection unit may be two-dimensional TOF (Time Of Flight) sensors or LiDAR (Light Detection And Ranging). If the first operator detection unit and the second operator detection unit are two-dimensional TOF sensors or LiDAR, the control unit may switch the operating authority based on posture information without using the operator's gaze information.

Also, the control unit may switch the operating authority based only on posture information without using gaze information.

Also, the control unit may be configured not to display the password screen in a state where the operator is not facing the front of the console and the operator's gaze is not directed toward the display unit.

Also, the control unit may be configured to control whether to display the password screen using both posture information and gaze information.

Also, the analysis unit may be configured to acquire posture information by a trained model that has learned to acquire posture information based on an image, and to acquire gaze information by a trained model that has learned to acquire gaze information based on an image.

Also, the analysis unit may be configured to acquire posture information and gaze information by performing image analysis of an optical image without using a trained model.

Also, the analysis unit and the control unit may be configured by a single processor.

Also, the first optical camera and the second optical camera do not have to be configured to be detachable.

Also, the X-ray imaging apparatus may be an apparatus that captures still images (a so-called general radiography apparatus) or an apparatus that images a subject during catheter treatment (a so-called X-ray angiography system).

Also, the first posture information and the second posture information do not have to be numerical information representing the degree of certainty that the operator is facing the front of the console. As long as the control unit can determine the operator's posture, the first posture information and the second posture information may be any kind of information, such as information indicating facing forward (e.g., "1") and information indicating not facing forward (e.g., "0" (zero)).

Also, the first posture information and the second posture information may be information on whether the operator is in front of the console. In this case, the control unit may switch the operating authority to the console when the operator has been in front of the console for a predetermined time.

Also, the first posture information and the second posture information may be information on whether the operator is in a posture capable of operating the console. Also, when the console is provided at a position where the X-ray operation unit is visible from an operator positioned in front of the console, the first posture information and the second posture information may be information on whether the operator is facing the direction of the X-ray operation unit.

Also, if the first posture information is equal to or greater than a predetermined first threshold and the first gaze information is equal to or greater than a predetermined second threshold, the control unit may switch the operating authority to the first console even if a predetermined time has not elapsed. The control unit may also perform similar control for the second console.

Also, the first gaze information and the second gaze information do not have to be numerical information representing the degree of certainty that the operator is looking at the display unit. As long as the control unit can determine the operator's gaze, the first gaze information and the second gaze information may be any kind of information, such as information indicating looking at the display unit (e.g., "1") and information indicating not looking at the display unit (e.g., "0" (zero)).

Also, the first gaze information and the second gaze information may be information representing whether the operator is looking at the X-ray imaging unit.

Also, when the first operator detection unit and the second operator detection unit are optical cameras, information such as the lab coats worn by the first operator and the second operator during operation may be acquired and stored in advance, and the control unit may be configured to switch the operating authority of the console based on the information such as the lab coats, along with the posture information and gaze information.

Also, when the first operator detection unit and the second operator detection unit are optical cameras, face information of the first operator and the second operator may be acquired and registered in advance, and the control unit may be configured to switch the operating authority of the console based on the operator's face information, along with the posture information and gaze information.

### [Aspects]

Those skilled in the art will understand that the exemplary embodiment described above is a specific example of the following aspects.

### (Item 1)

An X-ray imaging apparatus, comprising:

an X-ray imaging unit provided in an X-ray imaging room and having an X-ray irradiation unit that irradiates X-rays, an X-ray detection unit that detects the X-rays irradiated from the X-ray irradiation unit, and a top plate;

a first console provided in the X-ray imaging room and including a first operation unit for the X-ray imaging unit;

a second console provided outside the X-ray imaging room and including a second operation unit for the X-ray imaging unit;

a first operator detection unit that detects a first operator corresponding to the first console;

a second operator detection unit that detects a second operator corresponding to the second console;

an analysis unit that performs an analysis process on each of first detection information acquired by the first operator detection unit and second detection information acquired by the second operator detection unit to acquire first posture information of the first operator and second posture information of the second operator; and

a control unit that, based on the first posture information and the second posture information, performs control to switch an operating authority for enabling operation of the X-ray imaging unit between the first console and the second console.

Since the operating authority is switched between the first console and the second console based on the first posture information of the first operator and the second posture information of the second operator, the operation of the first console or the second console can be enabled without the first operator and the second operator performing an operation to switch the operating authority. As a result, it is possible to suppress the cumbersomeness of the operations of the first operator and the second operator when activating the operating authority. Furthermore, since the operating authority is switched between the first console and the second console based on the first posture information of the first operator and the second posture information of the second operator, it is possible to suppress unnecessary switching of the operating authority when an operator crosses in front of a console.

### (Item 2)

The X-ray imaging apparatus according to item 1, wherein the control unit performs control such that:

when the first posture information is information indicating that the first operator is facing the front of the first console, the operating authority is switched to the first console, and

when the second posture information is information indicating that the second operator is facing the front of the second console, the operating authority is switched to the second console.

When operating a console, an operator faces the front of the console. Therefore, by switching the operating authority to the first console when the first operator is facing the front of the first console, and switching the operating authority to the second console when the second operator is facing the front of the second console, it is possible to suppress the operating authority from being activated when a person who is not operating the console is in the vicinity of the console.

### (Item 3)

The X-ray imaging apparatus according to item 2, wherein, when the first operator is facing the front of the first console and the second operator is facing the front of the second console, the control unit performs control to switch the operating authority to the console, of the first console and the second console, for which an operator faced the front of the console at an earlier timing.

Since only the operating authority of the console that the operator faced first becomes active, it is possible to prevent the operating authorities of both the first console and the second console from being in an active state at the same time.

### (Item 4)

The X-ray imaging apparatus according to item 3, wherein, after the first operator has faced the front of the first console and the second operator has faced the front of the second console, if the first operator or the second operator moves away from the front of the console and a predetermined time has elapsed, the control unit performs control to switch the operating authority to the console, of the first console and the second console, that an operator is facing.

After either the first operator or the second operator moves away from the front of a console, the operating authority can be automatically switched to the console, of the first console and the second console, that an operator is facing. As a result, the operating authority is automatically switched without the operator who operates the console performing an operation to switch the operating authority, so the convenience (usability) for the operator can be improved.

### (Item 5)

The X-ray imaging apparatus according to any one of items 1 to 4, wherein:

the first console is provided with a first operating authority button for switching the operating authority between an active state and an inactive state, regardless of whether the operating authority has been granted,

the second console is provided with a second operating authority button for switching the operating authority between an active state and an inactive state, regardless of whether the operating authority has been granted, and

the control unit performs control such that:

when an operation to activate the operating authority is performed by the first operating authority button in a state where the operating authority is not granted to the first console, the operating authority is switched to the first console, and

when an operation to activate the operating authority is performed by the second operating authority button in a state where the operating authority is not granted to the second console, the operating authority is switched to the second console.

For example, when operating the X-ray imaging unit with multiple operators, in a configuration where the operating authority is switched based on whether an operator is facing the front of a console, if both the first operator and the second operator are facing their respective consoles, the operating authority cannot be switched to the other console unless one of the operators moves away from the front of their console. In this case, switching the operating authority between the first console and the second console becomes cumbersome, and the efficiency of operating the X-ray imaging unit decreases. Therefore, in the present invention, even when the operating authority is not granted, by performing an operation to activate the operating authority with the first operating authority button or performing an operation to activate the operating authority with the second operating authority button, the operating authority can be switched between the first console and the second console without moving away from the front of the console. As a result, when operating the X-ray imaging unit with multiple operators, it becomes possible to activate the operating authority at any necessary timing without moving away from the front of the console to switch the operating authority, thereby suppressing a decrease in the efficiency of operating the X-ray imaging unit.

### (Item 6)

The X-ray imaging apparatus according to any one of items 1 to 5, wherein:

the first operator detection unit is a first optical camera that captures a first optical image,

the second operator detection unit is a second optical camera that captures a second optical image, and

the analysis unit is configured to:

acquire the first posture information of the first operator appearing in the first optical image by analyzing the first optical image, and

acquire the second posture information of the second operator appearing in the second optical image by analyzing the second optical image.

The analysis unit can easily analyze the posture of the first operator and the posture of the second operator by analyzing the first optical image and the second optical image. As a result, the analysis unit can easily acquire the first posture information and the second posture information.

### (Item 7)

The X-ray imaging apparatus according to item 6, wherein:

the first console further includes a first display unit,

the second console further includes a second display unit,

the analysis unit:

acquires first gaze information of the first operator along with the first posture information by analyzing the first optical image, and

acquires second gaze information of the second operator along with the second posture information by analyzing the second optical image, and

the control unit performs control to:

switch the operating authority to the first console when the gaze of the first operator is directed toward the first display unit, and

switch the operating authority to the second console when the gaze of the second operator is directed toward the second display unit.

When operating a console, the operator's gaze will be directed toward the display unit. It is also not preferable to activate the operating authority of a console while the operator's gaze is not directed toward the display unit. In the present invention, since the operating authority is switched to the console toward which the operator's gaze is directed, it is possible to suppress the activation of the operating authority in a state where the operator's gaze is not directed toward the display unit.

### (Item 8)

The X-ray imaging apparatus according to any one of items 1 to 7, wherein:

the first console includes a first display unit that displays a first password screen for the first operator to enter a password to operate the X-ray imaging unit,

the second console includes a second display unit that displays a second password screen for the second operator to enter a password to operate the X-ray imaging unit, and

the control unit performs control such that:

in a state where there is no first operator facing the front of the first console, the first password screen is not displayed on the first display unit, and

in a state where there is no second operator facing the front of the second console, the second password screen is not displayed on the second display unit.

Since the first password screen is not displayed in a state where there is no first operator facing the front of the first console, and the second password screen is not displayed in a state where there is no second operator facing the front of the second console, it is possible to prevent a person other than the first operator and the second operator from unlocking the password via remote access or the like. As a result, the safety of the X-ray imaging apparatus can be improved.

### [Description of Reference Numerals]

10 X-ray imaging unit

11 X-ray irradiation unit

12 X-ray detection unit

13 Top plate

20 First console

21 First operation unit

22 First operating authority button

23 First operator detection unit

23a First optical camera

24 First display unit

30 Second console

31 Second operation unit

32 Second operating authority button

33 Second operator detection unit

33a Second optical camera

34 Second display unit

40 Analysis unit

50 Control unit

71 First optical image

72 Second optical image

73 First posture information

74 First gaze information

75 Operating authority

76 Second posture information

77 Second gaze information

80 First password screen

81 Second password screen

92 X-ray imaging room

100 X-ray imaging apparatus

1. An X-ray imaging apparatus, comprising:
an X-ray imaging unit provided in an X-ray imaging room and having an X-ray irradiation unit that irradiates X-rays, an X-ray detection unit that detects the X-rays irradiated from the X-ray irradiation unit, and a top plate;
a first console provided in the X-ray imaging room and including a first operation unit for the X-ray imaging unit;
a second console provided outside the X-ray imaging room and including a second operation unit for the X-ray imaging unit;
a first operator detection unit that detects a first operator corresponding to the first console;
a second operator detection unit that detects a second operator corresponding to the second console;
an analysis unit that performs an analysis process on each of first detection information acquired by the first operator detection unit and second detection information acquired by the second operator detection unit to acquire first posture information of the first operator and second posture information of the second operator; and
a control unit that, based on the first posture information and the second posture information, performs control to switch an operating authority for enabling operation of the X-ray imaging unit between the first console and the second console.

2. The X-ray imaging apparatus according to claim 1, wherein the control unit performs control such that:
when the first posture information is information indicating that the first operator is facing the front of the first console, the operating authority is switched to the first console, and
when the second posture information is information indicating that the second operator is facing the front of the second console, the operating authority is switched to the second console.

3. The X-ray imaging apparatus according to claim 2, wherein, when the first operator is facing the front of the first console and the second operator is facing the front of the second console, the control unit performs control to switch the operating authority to the console, of the first console and the second console, for which an operator faced the front of the console at an earlier timing.

4. The X-ray imaging apparatus according to claim 3, wherein, after the first operator has faced the front of the first console and the second operator has faced the front of the second console, if the first operator or the second operator moves away from the front of the console and a predetermined time has elapsed, the control unit performs control to switch the operating authority to the console, of the first console and the second console, that an operator is facing.

5. The X-ray imaging apparatus according to claim 1, wherein:
the first console is provided with a first operating authority button for switching the operating authority between an active state and an inactive state, regardless of whether the operating authority has been granted,
the second console is provided with a second operating authority button for switching the operating authority between an active state and an inactive state, regardless of whether the operating authority has been granted, and
the control unit performs control such that:
when an operation to activate the operating authority is performed by the first operating authority button in a state where the operating authority is not granted to the first console, the operating authority is switched to the first console, and
when an operation to activate the operating authority is performed by the second operating authority button in a state where the operating authority is not granted to the second console, the operating authority is switched to the second console.

6. The X-ray imaging apparatus according to claim 1, wherein:
the first operator detection unit is a first optical camera that captures a first optical image,
the second operator detection unit is a second optical camera that captures a second optical image, and
the analysis unit is configured to:
acquire the first posture information of the first operator appearing in the first optical image by analyzing the first optical image, and
acquire the second posture information of the second operator appearing in the second optical image by analyzing the second optical image.

7. The X-ray imaging apparatus according to claim 6, wherein:
the first console further includes a first display unit,
the second console further includes a second display unit,
the analysis unit:
acquires first gaze information of the first operator along with the first posture information by analyzing the first optical image, and
acquires second gaze information of the second operator along with the second posture information by analyzing the second optical image, and
the control unit performs control to:
switch the operating authority to the first console when the gaze of the first operator is directed toward the first display unit, and
switch the operating authority to the second console when the gaze of the second operator is directed toward the second display unit.

8. The X-ray imaging apparatus according to claim 1, wherein:
the first console includes a first display unit that displays a first password screen for the first operator to enter a password to operate the X-ray imaging unit,
the second console includes a second display unit that displays a second password screen for the second operator to enter a password to operate the X-ray imaging unit, and
the control unit performs control such that:
in a state where there is no first operator facing the front of the first console, the first password screen is not displayed on the first display unit, and
in a state where there is no second operator facing the front of the second console, the second password screen is not displayed on the second display unit.
